SEQUENCE LISTING (2 sequences)

SEQ ID NO: 1 - DNA, 1497 bp, Pseudomonas aeruginosa
atgaagaaggtttctacgcttgacctgttgttcgttgcgatcatgggtgtttcgccggccgcttttgccgccgacctgatcgacgtgtccaaactccccagcaaggctgcccagggcgcgcccggcccggtcaccttgcaagccgcggtcggcgctggcggtgccgacgaactgaaagcgatccgcagcacgaccctgcccaacggcaagcaggtcacccgctacgagcaattccacaacggcgtacgggtggtcggcgaagccatcaccgaagtcaagggtcccggcaagagcgtggcggcgcagcgcagcggccatttcgtcgccaacatcgctgccgacctgccgggcagcaccaccgcggcggtatccgccgagcaggtgctggcccaggccaagagcctgaaggcccagggccgcaagaccgagaatgacaaagtggaactggtgatccgcctgggcgagaacaacatcgcccaactggtctacaacgtctcctacctgattcccggcgagggactgtcgcggccgcatttcgtcatcgacgccaagaccggcgaagtgctcgatcagtgggaaggcctggcccacgccgaggcgggcggccccggcggcaaccagaagatcggcaagtacacctacggtagcgactacggtccgctgatcgtcaacgaccgctgcgagatggacgacggcaacgtcatcaccgtcgacatgaacagcagcaccgacgacagcaagaccacgccgttccgcttcgcctgcccgaccaacacctacaagcaggtcaacggcgcctattcgccgctgaacgacgcgcatttcttcggcggcgtggtgttcaaactgtaccgggactggttcggcaccagcccgctgacccacaagctgtacatgaaggtgcactacgggcgcagcgtggagaacgcctactgggacggcacggcgatgctcttcggcgacggcgccaccatgttctatccgctggtgtcgctggacgtggcggcccacgaggtcagccacggcttcaccgagcagaactccgggctgatctaccgcgggcaatcaggcggaatgaacgaagcgttctccgacatggccggcgaggctgccgagttctatatgcgcggcaagaacgacttcctgatcggctacgacatcaagaagggcagcggtgcgctgcgctacatggaccagcccagccgcgacgggcgatccatcgacaacgcgtcgcagtactacaacggcatcgacgtgcaccactccagcggcgtgtacaaccgcgcgttctacctgttggccaattcgccgggctgggatacccgcaaggccttcgaggtgttcgtcgacgccaaccgctactactggaccgccaccagcaactacaacagcggcgcctgcggggtgattcgctcggcgcagaaccgcaactactcggcggctgacgtcacccgggcgttcagcaccgtcggcgtgacctgcccgagcgcgttgtaa

SEQ ID NO: 2 - protein, 301 aa, Pseudomonas aeruginosa
AEAGGPGGNQKIGKYTYGSDYGPLIVNDRCEMDDGNVITVDMNSSTDDSKTTPFRFACPTNTYKQVNGAYSPLNDAHFFGGVVFKLYRDWFGTSPLTHKLYMKVHYGRSVENAYWDGTAMLFGDGATMFYPLVSLDVAAHEVSHGFTEQNSGLIYRGQSGGMNEAFSDMAGEAAEFYMRGKNDFLIGYDIKKGSGALRYMDQPSRDGRSIDNASQYYNGIDVHHSSGVYNRAFYLLANSPGWDTRKAFEVFVDANRYYWTATSNYNSGACGVIRSAQNRNYSAADVTRAFSTVGVTCPSAL

(19) United States
(12) Patent Application Publication
(10) Pub. No.: US 2012/0295294 A1
(43) Pub. Date: Nov. 22, 2012

(54) ENZYMATIC METHOD FOR PREPARING ASPARTAM

(75) Inventors: Zhuying WANG, Monmouth Junction, NJ (US); Jian SUN, Nanjing (CN); Xiangqun LI, Lutherville-Timonium, MD (US); Fang Liang ZHANG, Fanwood, NJ (US)

(73) Assignee: GENSCRIPT NANJING, Nanjing (CN)

(21) Appl. No.: 13/473,993

(22) Filed: May 17, 2012

Related U.S. Application Data
(60) Provisional application No. 61/487,375, filed on May 18, 2011.

(51) Int. Cl. C12P 21/02; G01N 27/62

(52) U.S. Cl. 435/23; 435/68.1

(57) ABSTRACT

An improved method is described for the synthesis of aspartame using a condensation reaction between the alpha-carboxyl group of the L-aspartic acid and the amino group of the methyl L-phenylalaninate catalyzed by an enzyme. The method allowed efficient and cost effective production of aspartame. A method of identifying an enzyme useful for preparing aspartame is also described.

### CROSS-REFERENCE TO RELATED APPLICATIONS

This application claims the benefit under 35 U.S.C. §119(e) of U.S. Provisional Application No. 61/487,375, filed May 18, 2011, the entire disclosure of which is hereby incorporated herein by reference.

### FIELD OF THE INVENTION

This invention relates to an improved enzymatic method for the synthesis of aspartame using unprotected amino acids as substrates under conditions where a sufficiently high yield of aspartame is obtained, and other related methods.

### BACKGROUND OF THE INVENTION

Aspartame is a methyl ester of the dipeptide of amino acids L-aspartic acid (L-Asp) and L-phenylalanine (L-Phe). It is approximately 200 times sweeter than sucrose (table sugar), and has been widely used as an artificial sweetener to substitute table sugar due to its negligible caloric contribution. Magnuson B A et al. (2007), “Aspartame: a safety evaluation based on current use levels, regulations, and toxicological and epidemiological studies,” Critical Reviews in Toxicology 37 (8): 629-727.

Aspartame has been synthesized by three approaches: chemical synthesis, enzymatic synthesis and mixed-method synthesis. During the chemical synthesis, the two carboxyl groups of aspartic acid are joined to form an anhydride, and the amino group is protected by a protection group to prevent further reactions of the amino group. Methylated phenylalanine is then reacted with the N-protected aspartic anhydride. The chemical removal of the N-blocking group by acid hydrolysis produces the final product aspartame. But this technique has the drawback of producing a byproduct of the bitter tasting β-form with the side chain carboxyl group from aspartic acid linked to phenylalanine. This technique can be improved by protecting the side carboxyl group of aspartic acid. However, the additional protecting procedure, subsequent activation of the alpha-carboxyl group from aspartic acid and the final removal of the protection group increases the production costs significantly.

A process using an enzyme from Bacillus thermoproteolyticus to catalyze the condensation of the chemically protected amino acids (mixed method synthesis) can produce high yields without the β-form byproduct due to the specificity of the enzyme. However, the drawback of this technique is that the amino acids need to be properly protected to prevent other side reactions. Yagasaki et al. (2008), “Synthesis and application of dipeptides; current status and perspectives,” Applied Microbiology and Biotechnology 81 (1): 13-22. A variant of this method, the enzymatic synthesis method, uses unprotected aspartic acid, but produces low yields that cannot be used commercially. Mixed methods for directly producing aspartyl-phenylalanine by enzymatic means, followed by chemical methylation, to produce aspartame have also been tried, but not scaled for industrial production. Yagasaki et al. (2008), above.

U.S. Pat. No. 4,916,062 described an enzymatic method for the synthesis of aspartame by condensation of L-aspartic acid with methyl L-phenylalaninate using an enzyme isolated from the multi-enzyme SP component of Micrococcus caseolyticus Strain I 194. However, only about 0.041 mmole aspartame was obtained from 20 mmoles each of the substrates, a yield that is too low for the method to be useful for commercial purposes.

In general, the conventional methods for aspartame production have some major drawbacks. For example, both of the chemical synthesis method and the mixed method synthesis require protection of the amino acid substrates prior to condensation and removal of the protection after the condensation, thus are cumbersome and costly. Although the enzymatic synthesis method does not require the protection and the removal of the protection, it is not efficient enough for industrial applications.

There is an unmet need for a simple, efficient and cost effective process for the production of aspartame. Embodiments of the present invention relate to such a process and related methods.

### BRIEF SUMMARY OF THE INVENTION

It has been surprisingly discovered that, under certain conditions, aspartame can be produced at a sufficiently high yield by an enzyme catalyzed condensation of L-aspartic acid with methyl L-phenylalaninate (methyl L-Phe), without the needs to protect the amino acid substrates prior to the condensation and to remove the protection after the condensation.

In one general aspect, the present invention relates to a method of preparing aspartame. The method comprises:

(1) providing a reaction mixture having a pH of about 2.0 to 5.5, the reaction mixture comprising:
(a) an effective amount of L-aspartic acid having no protection of either of its amino group and side chain carboxyl group;
(b) an effective amount of methyl L-phenylalaninate;
(c) a catalytically effective amount of an enzyme capable of catalyzing a condensation reaction between the alpha-carboxyl group of the L-aspartic acid and the amino group of the methyl L-phenylalaninate;
(d) about 20% to 90% (v/v) of an organic solvent; and
(e) an aqueous solution; and

(2) incubating the reaction mixture at a temperature of about 25° C. to 60° C. to obtain aspartame by the condensation reaction.

In another general aspect, the present invention relates to a method of preparing aspartame, comprising:

(1) providing a reaction mixture having a pH of about 3.0 to 4.5, the reaction mixture comprising:
(a) an effective amount of L-aspartic acid;
(b) an effective amount of methyl L-phenylalaninate, wherein the molar ratio of the effective amount of L-aspartic acid to the effective amount of methyl L-phenylalaninate is about 1:20 to 1:4;

(c) a catalytically effective amount of an enzyme comprising the amino acid sequence of SEQ ID NO:2;
(d) about 30% to 70% (v/v) of an organic solvent; and
(e) an aqueous solution; and

(2) incubating the reaction mixture at a temperature of about 30° C. to 50° C. to obtain aspartame by a condensation reaction between the alpha-carboxyl group of the L-aspartic acid and the amino group of the methyl L-phenylalaninate catalyzed by the enzyme.

In yet another general aspect, the present invention relates to a method of identifying an enzyme useful for preparing aspartame, comprising:

(1) providing a reaction mixture having a pH of about 2.0 to 5.5, the reaction mixture comprising:
(a) an effective amount of L-aspartic acid having no protection of either of its amino group and side chain carboxyl group;
(b) an effective amount of methyl L-phenylalaninate;
(c) an effective amount of a test enzyme;
(d) about 20% to 90% (v/v) of an organic solvent; and
(e) an aqueous solution;

(2) incubating the reaction mixture at a temperature of about 25° C. to 60° C.;

(3) measuring the amount of aspartame in the reaction mixture after the incubating step; and

(4) identifying the test enzyme as useful for preparing aspartame if yield of aspartame reaches a threshold.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The foregoing summary, as well as the following detailed description of the invention, will be better understood when read in conjunction with the appended drawing. For the purpose of illustrating the invention, there is shown in the drawing embodiments of the invention. It should be understood, however, that the invention is not limited to the precise arrangements and instrumentalities shown.

In the drawings:

FIG. 1 schematically illustrates the enzymatic synthesis of aspartame according to an embodiment of the present invention;

FIG. 2 shows spectra of mass spectrum analysis of the supernatant of a reaction production according to an embodiment of the present invention (A), and an aspartame standard (B); and

FIG. 3 shows the HPLC analysis of a reaction product according to an embodiment of the present invention.

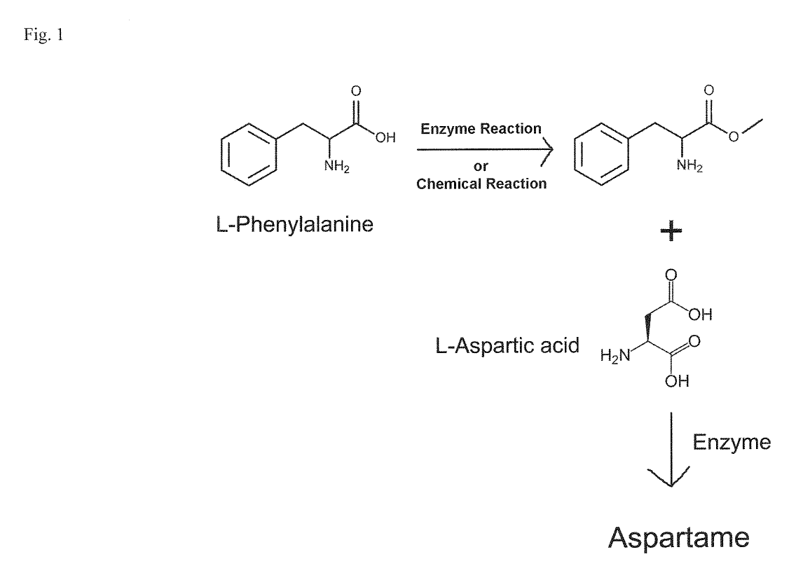

### DETAILED DESCRIPTION OF THE INVENTION

Unless defined otherwise, all technical and scientific terms used herein have the same meaning as commonly understood to one of ordinary skill in the art to which this invention pertains. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

It was previously reported that PT121 protease, an organic solvent-stable protease isolated from Pseudomonas aeruginosa, catalyzed the synthesis of aspartame precursor Cbz-Asp-Phe-NH₂. Tang et al. (2008), “Screening and isolation of an organic solvent-tolerant bacterium for high-yield production of organic solvent-stable protease,” Bioresource Technology 99 (15): 7388-7392. In particular, in the presence of 50% dimethyl sulfoxide (DMSO) at a temperature of 37° C., the enzyme catalyzed the condensation of an N-protected aspartic acid Cbz-Asp-OH (0.05M) with L-Phe-NH₂ (0.1M), resulting in high yield of aspartame precursor Cbz-Asp-Phe-NH₂. The amino group on Asp was protected, e.g., by Cbz (carboxybenzyl), to prevent it from reacting with the carboxyl group of Phe to produce side products.

The PT121 protease catalyzes a condensation reaction between the alpha-carboxyl group of one amino acid, such as the L-aspartic acid, and the amino group of another amino acid, such as L-phenylalanine. Previous efforts using unprotected Asp and methyl L-phenylalaninate as substrates for the PT121 protease had failed to result in direct formation of aspartame (personal communication with B. F. He).

It has now been surprisingly discovered that under certain conditions, the PT121 protease catalyzed specific condensation of the alpha-carboxyl group of L-Asp with the amino group of methyl L-Phe, resulting in a significantly higher yield of aspartame, i.e., more than 100 times higher, than that obtained with the previous enzymatic synthesis method described in U.S. Pat. No. 4,916,062.

According to embodiments of the present invention, under suitable assay conditions, such as the presence of adequate amount of organic solvent and low pH in the reaction mixture, an enzyme is capable of selectively and specifically condensing the alpha-carboxyl group of L-aspartic acid with the amino group of methyl L-phenylalaninate to directly produce aspartame at a sufficiently high yield. In other words, the enzyme uses methyl L-phenylalaninate as the only or major amino group donor to condense with the alpha-carboxyl group of L-aspartic acid to form a peptide bond. It does not use or uses very little L-aspartic acid as the amino group donor even though the amino group of L-aspartic acid is free (unprotected) for reaction. It also does not condense or condenses very little the amino group of methyl L-phenylalaninate with the side chain carboxyl group of L-aspartic acid, even though the side chain carboxyl group is also unprotected.

Accordingly, a method according to an embodiment of the present invention produces aspartame at a significantly high yield without major side products even though neither the amino group nor the side chain carboxyl group of L-aspartic acid is protected. Such a method simplifies the production process and significantly reduces the production costs.

It has been discovered in the present invention that the pH of the reaction mixture significantly affects the yield of aspartame. For example, the yield was increased about 2 folds when the pH was increased from 3 to 4, but was reduced more than two folds when the pH was further increased from 4 to 4.5 (see Table 4 below). Thus, according to embodiments of the present invention, the reaction mixture has a pH of about 2.0 to 5.5, preferably 3.0 to 4.5. Examples of pH of the reaction mixture suitable for the present invention include, but are not limited to, a pH of about 2.0, 3.0, 3.5, 4.0, 4.5, 5.0, 5.5, etc.

According to embodiments of the present invention, a reaction mixture is first provided by combining various components in a container. The reaction mixture is then incubated at a temperature of about 25° C. to 60° C., preferably about 30° C. to 50° C., and most preferably about 43° C., to allow production of aspartame by an enzyme catalyzed condensation reaction between the two amino acid substrates, L-Asp and methyl L-Phe.

A reaction mixture according to an embodiment of the present invention comprises:

(a) an effective amount of L-aspartic acid;

(b) an effective amount of methyl L-phenylalaninate;

(c) a catalytically effective amount of an enzyme capable of catalyzing a condensation reaction between the alpha-carboxyl group of the L-aspartic acid and the amino group of the methyl L-phenylalaninate;

(d) about 20% to 90% (v/v) of an organic solvent; and

(e) an aqueous solution.

Aspartame is chiral. Preferably, only L-formed amino acids are used as the substrates. L-Asp and L-Phe can be obtained by fermentation or from commercial sources. Preferably, the L-Asp has no protection of either of its amino group and side chain carboxyl group. Methyl L-Phe, the methyl ester of phenylalanine, can be obtained using methods known in the art. For example, it can be obtained by treating L-Phe with methanol in the presence hydrochloric acid to cause an ester reaction with the acid group on the phenylalanine. It can also be obtained by enzymatic esterification of L-Phe.

It has been discovered in the present invention that the conversation rate of aspartame is affected by the molar ratio of the effective amount of L-aspartic acid and the effective amount of methyl L-phenylalaninate used in the reaction mixture (see Table 2 below). Accordingly, the preferred molar ratio of L-Asp to methyl L-Phe is about 1:20 to 1:4, preferably about 1:10. Examples of the molar ratio of L-Asp to methyl L-Phe in the reaction mixture suitable for the present invention include, but are not limited to 1:20, 1:16, 1:12, 1:10, 1:8, 1:6, 1:4, etc.

It has been discovered in the present invention that the amount of organic solvent present in the reaction mixture significantly affects the yield of aspartame. For example, no aspartame was detected from a reaction mixture containing 0% or 15% DMSO, the yield of aspartame initially increased with increased concentration of organic solvent, but then decreased sharply with further increase of the concentration of the organic solvent (Table 1). According to embodiments of the present invention, the reaction mixture comprises about 20% to 90% (v/v), preferably about 30% to 70% of an organic solvent. Examples of the amount of an organic solvent in the reaction mixture suitable for the present invention include, but are not limited to, about 30%, 40%, 50%, 60%, 70%, etc., preferably about 60%, by volume.

The reaction mixture can include a single organic solvent or a mixture of two or more organic solvents. Examples of organic solvents suitable for the present invention include, but are not limited to, dimethylformamide (DMF), methanol, ethanol, dimethyl sulfoxide (DMSO), ethyl acetate, n-butyl acetate, n-butanol, isobutanol, isoamyl alcohol, dichloromethane, chloroform, n-hexane, cyclohexane, n-octane, acetonitril, an ether and tetrahydrofuran (THF), etc., preferably methanol or DMSO.

According to embodiments of the present invention, the reaction mixture also contains an aqueous solution, such as a sodium acetate buffer, a Tris HCl buffer, H₂O, etc.

The reaction mixture can be a one-phase system with the organic solvent and the aqueous solution in one phase. It can also be a multi-phase system, with one phase being an aqueous phase and the other organic phase(s).

The catalytically effective amount of an enzyme is the amount of the enzyme that is effective to catalyze the condensation of L-aspartic acid with methyl L-phenylalaninate to produce the desired amount of aspartame. The effective amount depends on factors, such as the desired amount of aspartame to be produced, the specific activity of the enzyme, the effective amount of L-aspartic acid and the effective amount of methyl L-phenylalaninate used in the reaction mixture, etc.

An enzyme suitable for the present invention can be any enzyme capable of catalyzing a condensation reaction between the alpha-carboxyl group of the L-aspartic acid and the amino group of the methyl L-phenylalaninate. Such enzymes include those that have been previously identified as to require protection of the amino group of Asp for the specific production of aspartame. Under reaction conditions according to embodiments of the present invention, those enzymes can now more specifically catalyze the condensation reaction between the alpha-carboxyl group of the L-aspartic acid and the amino group of the methyl L-phenylalaninate, no longer requiring protection of the amino group of L-Asp.

In a preferred embodiment, the enzyme used in the present invention is the PT121 protease, which comprises the amino acid sequence of SEQ ID NO: 2. It can be encoded by a nucleic acid comprising SEQ ID NO: 1.

In another embodiment of the present invention, the suitable enzyme is a homolog or derivative of the PT121 protease, which comprises an amino acid sequence having at least about 80% sequence identity to SEQ ID NO:2. The enzyme can have at least about 80%, 85%, 90%, 95% or 100% sequence identity to SEQ ID NO:2. Such homologs or derivatives of the PT121 protease can be made and identified in view of the present disclosure.

For example, a homolog of the PT121 protease can be identified from other Pseudomonas species, preferably other Pseudomonas aeruginosa strains or isolates by a combination of sequence analysis and functional verification, using methods known in the art in combination with a method of the present invention described below.

Derivatives of the PT121 protease, such as those containing one or more substitutions, deletions and additions (or insertions) in the amino acid sequence of PT121 protease, can be made using conventional molecular biology tools in view of the disclosed sequence of PT121 protease. It is well known in the art that some alterations in a polypeptide sequence, especially those amino acids that are away from the active site, do not affect the functional properties of the protein or enzyme. It is also well known in the art that some alterations in a polypeptide sequence, especially those amino acids that in or close to the active site, can improve the functional properties of the protein or enzyme. Such derivatives of the PT121 protease can be made by genetic engineering, and screened or assayed using a method of the present invention described below.

According to an embodiment of the present invention, an enzyme useful for preparing aspartame can be identified from a screening method, comprising:

(1) providing a reaction mixture having a pH of about 2.0 to 5.5, the reaction mixture comprising:
(a) an effective amount of L-aspartic acid having no protection of either of its amino group and side chain carboxyl group;
(b) an effective amount of methyl L-phenylalaninate;
(c) an effective amount of a test enzyme;
(d) about 20% to 90% (v/v) of an organic solvent; and
(e) an aqueous solution;

(2) incubating the reaction mixture at a temperature of about 25° C. to 60° C.;

(3) measuring the amount of aspartame in the reaction mixture after the incubating step; and

(4) identifying the test enzyme as useful for preparing aspartame if yield of aspartame reaches a threshold.

The reaction mixture is identical to that used in the method for preparing aspartame, except that a test enzyme, instead of an enzyme with known activity to catalyze the desired condensation reaction, is used in the screening method.

After the incubating step, the amount of aspartame in the reaction mixture or the reaction product can be measured using methods known to those skilled in the art, such as mass spectrum analysis, chromatography analysis, etc. Yield of aspartame can be calculated based on the amount of the measured aspartame and the amount of substrates originally included in the reaction mixture.

The test enzyme is identified as useful for preparing aspartame if the yield of aspartame reaches a threshold. The threshold is a yield of aspartame pre-defined by the researcher. It can be, for example, 10%, 20%, 30%, 40%, 50%, 60%, 70%, 80%, or 90%, molar ratio, based on the effective amount of L-Asp used in the reaction mixture.

The test enzyme can be any enzyme. Preferably, it is a homolog or derivative of the PT121 protease having at least about 60% amino acid identity to the sequence SEQ ID No.2.

The enzyme or test enzyme can be prepared by conventional methods in view of the present disclosure, such as by purification from a cell endogenously or natively producing the enzyme, or from a cell recombinantly producing the enzyme. The native host cell can be, e.g., a Pseudomonas cell, preferably a Pseudomonas aeruginosa cell. The recombinant host cell can be, indiscriminately, a bacterium, a fungus or a yeast.

It is readily apparent to those skilled in the art that methods of the present invention can also be conducted using L-Asp having protected amino group and/or protected side chain carboxyl group. Even though such protection may not be required, using protected L-Asp may further reduce or eliminate the minor production of side products by the enzyme. The amino group protection moieties include, but are not are limited to, Boc-, Bpoc, Fmoc, CBZ-, trityl-, Nps-, etc.

Various embodiments of the invention have now been described. It is to be noted, however, that this description of these specific embodiments is merely illustrative of the principles underlying the inventive concept. It is therefore contemplated that various modifications of the disclosed embodiments will, without departing from the spirit and scope of the invention, be apparent to persons skilled in the art.

The following specific examples of the methods of the invention are further illustrative of the nature of the invention, it needs to be understood that the invention is not limited thereto.

### EXAMPLES

### Example 1

### Preparation of Crude Enzyme Powder of PT121 Protease

The PT121 strain isolate described in Tang et al. (2008) was used for the preparation of crude enzyme powder of PT121 protease. The PT121 isolate was characterized to belong to Pseudomonas genus and showed high degree of similarity (>98%) to Pseudomonas aeruginosa by 16S rRNA analysis.

The PT121 isolate was grown in the following medium:

Tryptone 10 g/L

(NH₄)SO₄ 4 1.0 g/L

KH₂PO₄ 0.5 g/L

MgSO₄ 0.3 g/L

CaCl₂ 1.0 g/L

NaCl 1.0 g/L

Glycerol 6.3 g/L

pH 7.0

The strain was cultured by shaking at 37° C. at 180 rpm for 72 hour (h). The culture was centrifuged at 10,000 rpm for 15 min at 4° C. The supernatant was separated and defined as crude enzyme solution. The crude enzyme solution was lyophilized to make the crude enzyme powder.

### Example 2

### The Effect of Organic Solvent Concentration on APM Yield

Different concentrations of organic solvent were included in reaction mixtures to study the effect of organic solvent concentration on the yield of aspartame (APM yield).

The synthesis of aspartame was carried out using L-Asp and methyl L-Phe (L-Phe-OMe) as substrates with 15 mg crude enzyme power: 30 mM L-Asp was used with 344 mM L-Phe-OMe in a 10 ml reaction mixture. The reaction was performed in 0.1M sodium Tris-HCl buffer solution at pH 4.0 with 0%, 15%, 30%, 60% or 70% DMSO, by volume, and was incubated at 37° C. overnight (15 h).

The yields of APM are listed in Table 1:

| DMSO (v/v) | APM yield (mg/ml) |
|---|---|
| 0% | 0 |
| 15% | 0 |
| 30% | 0.26 |
| 60% | 1.04 |
| 70% | 0.37 |

### Example 3

### The Effect of Substrate Concentration on APM Yield

Different concentrations of L-Asp were included in reaction mixtures with mixed amount of L-Phe-OMe to study the effect of substrate concentration on the APM yield.

The synthesis of aspartame was carried out using L-Asp and L-Phe-OMe as substrates with 15 mg crude enzyme power. L-Asp at 30 mM, 50 mM, 60 mM or 70 mM was used respectively with 344 mM L-Phe-OMe in a 10 ml reaction mix. The reaction was performed in 0.1 M sodium acetate-acetic acid buffer solution at pH 4.0 with 60% DMSO at 37° C. overnight (15 h).

The yields of APM are listed in Table 2:

| L-Asp conc. | APM yield (mM/ml) | Conversion rate based on L-Asp (%) |
|---|---|---|
| 30 mM | 9.3 | 30.9 |
| 50 mM | 13.3 | 26.6 |
| 60 mM | 15.3 | 25.3 |
| 70 mM | 16.7 | 23.9 |

### Example 4

### The Effect of Reaction Temperature on APM Yield

Different reaction temperatures were tested to study the effect of reaction temperature on the APM yield.

The reaction was carried out in 37° C., 43° C. 47° C., 50° C. or 60° C. overnight (15 h). The 10 ml reaction mixture contained: 30 mM L-Asp, 344 mM L-Phe-OMe, 15 mg crude enzyme power, 0.1 M sodium acetate-acetic acid and 60% (v/v) DMSO, at pH 4.0.

The yields of APM are listed in Table 3:

| | | Conversion rate |
|---|---|---|
| Temperature (° C.) | APM yield (mM/ml) | based on L-Asp (%) |
| 37 | 2.73 | 11.4 |
| 43 | 4.17 | 17.4 |
| 47 | 4.01 | 16.7 |
| 50 | 3.68 | 15.4 |
| 60 | 0.41 | 1.69 |

### Example 5

### The Effect of pH on AMP Yield

Different pHs were tested to study the effect of pH in the reaction mixture on the APM yield.

Sodium acetate-acetic acid buffer solution (0.1M) with pH adjusted to 3, 3.5, 4 or 4.5 was included in the reaction mixture for aspartame synthesis. The reaction mixture further included 24 mM L-Asp, 344 mM L-Phe-OMe, 15 mg crude enzyme powder and 60% DMSO, and was incubated at 43° C. overnight (15 h).

The yields of APM are listed in Table 4:

| pH | APM yield (mM/ml) | Conversion rate compared to L-Asp (%) |
|---|---|---|
| 3 | 2.0 | 8.3 |
| 3.5 | 2.6 | 11.0 |
| 4 | 3.9 | 16.3 |
| 4.5 | 1.8 | 7.5 |

### Example 6

### Preparation of Aspartame

L-aspartic acid (30 mM) and 344 mM of methyl L-phenylalaninate were mixed in 10 ml of 0.1M sodium Tris-HCl buffer (pH 4.0) containing 60% DMSO. PT121 crude enzyme power (15 mg) was added to the mixture and the reaction mixture was stirred at 37° C. overnight (15 hours).

The concentration of aspartame reached 9.3 mM as analyzed by HPLC. The aspartame yield was 30.9% based on L-aspartic acid conversion.

### Example 7

### Measuring Aspartame in the Reaction Product by MS Analysis

A reaction mixture was prepared, which contained: 0.6 (v/v) methanol solution, 30 mM L-Asp, 344 mM L-Phe-OMe, and 15 mg crude enzyme powder, at pH 4.0. The reaction mixture was incubated at 43° C. overnight (15 h).

The reaction product was centrifuged to collect the supernatant. After passing the supernatant through a 0.22 μm membrane for clean up, the filtered supernatant was analyzed by mass spectrum (MS).

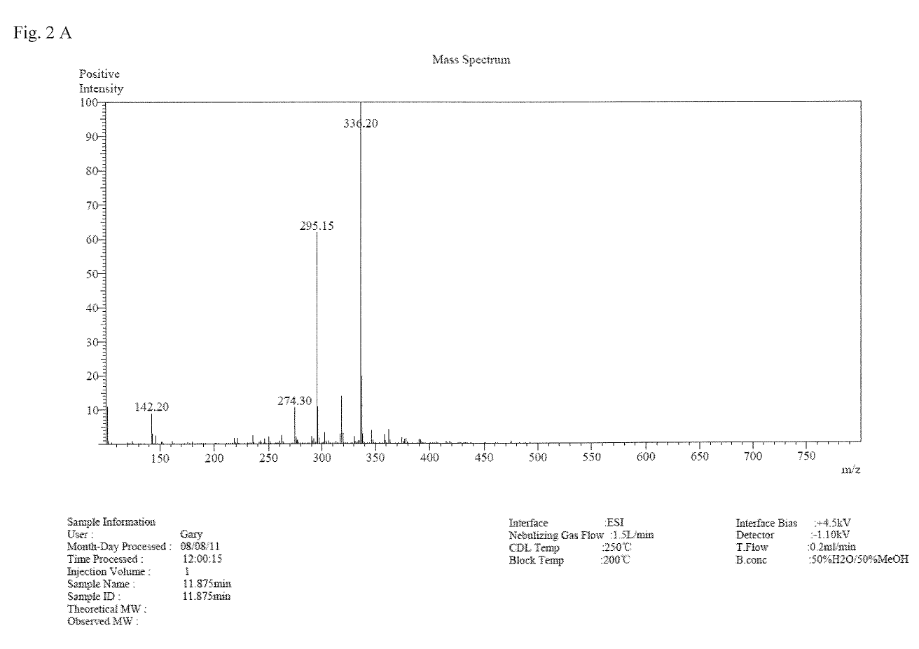

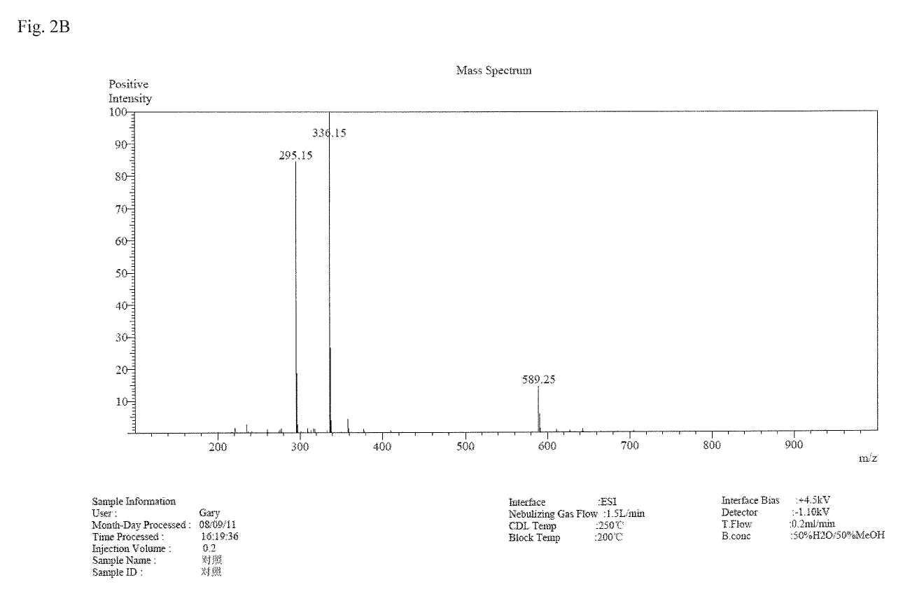

The MS spectrum for the reaction production (FIG. 2A) contained aspartame, which matched well with the MS spectrum for an aspartame standard (FIG. 2B).

### Example 8

### Measuring Aspartame in the Reaction Product by HPLC Analysis

The reaction product was dissolved in 50% methanol (v/v) at pH 4.0. It was separated and analyzed on Shimadzu® LC-2010A HT using 5%-65% gradient of H₂O (containing 0.05% TFA) to acetonitrile (containing 0.05% TFA) under a flow-rate of 1 ml/min.

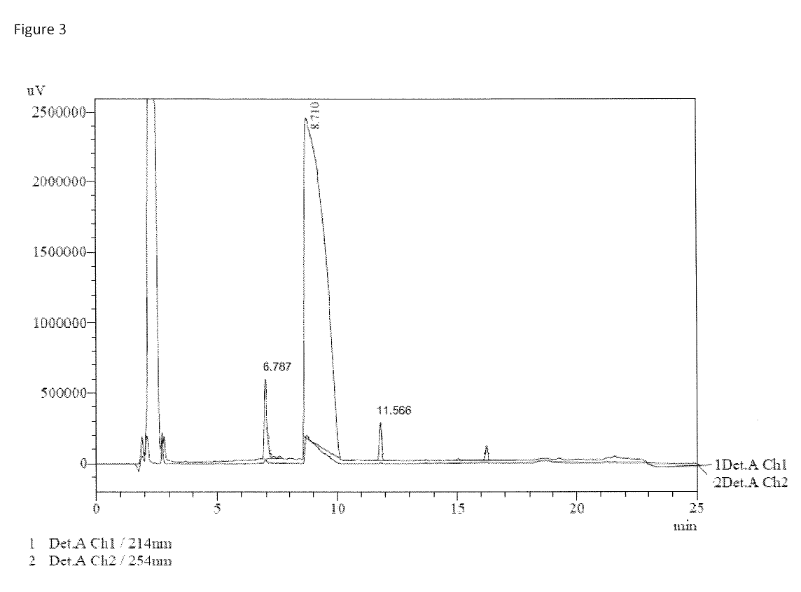

The products were monitored at 214 nm and 254 nm. Aspartame and the two substrates, L-Asp and L-Phe-OMe had retention time of 11.6 min, 6.9 min and 8.7 min respectively. As shown in FIG. 3, the reaction product contained aspartame.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of preparing aspartame, comprising
(1) providing a reaction mixture having a pH of about 2.0 to 5.5, the reaction mixture comprising:
(a) an effective amount of L-aspartic acid having no protection of either of its amino group and side chain carboxyl group;
(b) an effective amount of methyl L-phenylalaninate;
(c) a catalytically effective amount of an enzyme capable of catalyzing a condensation reaction between the alpha-carboxyl group of the L-aspartic acid and the amino group of the methyl L-phenylalaninate;
(d) about 20% to 90% (v/v) of an organic solvent; and
(e) an aqueous solution; and
(2) incubating the reaction mixture at a temperature of about 25° C. to 60° C. to obtain aspartame by the condensation reaction.

2. The method of claim 1, wherein the enzyme comprises an amino acid sequence having at least about 80% sequence identity to SEQ ID NO:2.

3. The method of claim 2, wherein the enzyme comprises an amino acid sequence having at least about 90% sequence identity to SEQ ID NO:2.

4. The method of claim 3, wherein the enzyme comprises the amino acid sequence of SEQ ID NO:2.

5. The method of claim 1, wherein the reaction mixture comprises about 30% to 70% (v/v) of an organic solvent selected from the group consisting of dimethylformamide, methanol, ethanol, dimethyl sulfoxide, ethyl acetate, n-butyl acetate, n-butanol, isobutanol, isoamyl alcohol, dichloromethane, chloroform, n-hexane, cyclohexane, n-octane, acetonitril, an ether and tetrahydrofuran.

6. The method of claim 5, wherein the reaction mixture comprises about 60% (v/v) dimethyl sulfoxide or methanol.

7. The method of claim 1, wherein the reaction mixture has a pH of about 3.0 to 4.5.

8. The method of claim 7, wherein the reaction mixture has a pH of about 4.0.

9. The method of claim 1, wherein the molar ratio of the effective amount of L-aspartic acid to the effective amount of methyl L-phenylalaninate is about 1:20 to 1:4.

10. The method of claim 1, wherein the reaction mixture is incubated at a temperature of about 30° C. to 50° C.

11. The method of claim 10, wherein the reaction mixture is incubated at a temperature of about 43° C.

12. A method of preparing aspartame, comprising:
(1) providing a reaction mixture having a pH of about 3.0 to 4.5, the reaction mixture comprising:
(a) an effective amount of L-aspartic acid;
(b) an effective amount of methyl L-phenylalaninate, wherein the molar ratio of the effective amount of L-aspartic acid to the effective amount of methyl L-phenylalaninate is about 1:20 to 1:4;
(c) a catalytically effective amount of an enzyme comprising the amino acid sequence of SEQ ID NO:2;
(d) about 30% to 70% (v/v) of an organic solvent; and
(e) an aqueous solution; and
(2) incubating the reaction mixture at a temperature of about 30° C. to 50° C. to obtain aspartame by a condensation reaction between the alpha-carboxyl group of the L-aspartic acid and the amino group of the methyl L-phenylalaninate catalyzed by the enzyme.

13. The method of claim 12, wherein the reaction mixture comprises about 60% (v/v) dimethyl sulfoxide or methanol, has a pH of about 4.0, and is incubated at a temperature of about 43° C.

14. The method of claim 12, wherein the L-aspartic acid is protected at its amino group.

15. A method of identifying an enzyme useful for preparing aspartame, comprising:
(1) providing a reaction mixture having a pH of about 2.0 to 5.5, the reaction mixture comprising:
(a) an effective amount of L-aspartic acid having no protection of either of its amino group and side chain carboxyl group;
(b) an effective amount of methyl L-phenylalaninate;
(c) an effective amount of a test enzyme;
(d) about 20% to 90% (v/v) of an organic solvent; and
(e) an aqueous solution;
(2) incubating the reaction mixture at a temperature of about 25° C. to 60° C.;
(3) measuring the amount of aspartame in the reaction mixture after the incubating step; and
(4) identifying the test enzyme as useful for preparing aspartame if yield of aspartame reaches a threshold.

16. The method of claim 15, wherein the test enzyme comprises an amino acid sequence having at least about 60% sequence identity to SEQ ID NO:2.

17. The method of claim 15, wherein the reaction buffer comprises about 30% to 70% (v/v) of an organic solvent selected from the group consisting of dimethylformamide, methanol, ethanol, dimethyl sulfoxide, ethyl acetate, n-butyl acetate, n-butanol, isobutanol, isoamyl alcohol, dichloromethane, chloroform, n-hexane, cyclohexane, n-octane, acetonitril, an ether and tetrahydrofuran.

18. The method of claim 15, wherein the reaction buffer has a pH of about 3.0 to 4.5.

19. The method of claim 15, wherein the reaction mixture is incubated at a temperature of about 30° C. to 50° C.

20. The method of claim 15, wherein the molar ratio of the effective amount of L-aspartic acid to the effective amount of methyl L-phenylalaninate is about 1:20 to 1:4, the reaction mixture comprises about 60% (v/v) dimethyl sulfoxide or methanol, has a pH of about 4.0, and is incubated at a temperature of about 43° C.
